(19) United States
(12) Patent Application Publication
(10) Pub. No.: US 2024/0060052 A1
(43) Pub. Date: Feb. 22, 2024

(54) Artificial Skeletal Muscle Tissue

(71) Applicant: VALO HEALTH, INC., Boston, MA (US)

(72) Inventors: Roozbeh Aschar-Sobbi, New York, NY (US); Isabella Pallotta, New York, NY (US)

(21) Appl. No.: 18/500,552

(22) Filed: Nov. 2, 2023

Related U.S. Application Data
(62) Division of application No. 16/952,267, filed on Nov. 19, 2020.
(60) Provisional application No. 62/940,976, filed on Nov. 27, 2019.

(51) Int. Cl. C12N 5/071; C12N 5/077; C12M 3/00; C12M 1/32; C12M 1/34; C12M 1/42; G01N 33/50

(52) U.S. Cl. CPC C12N 5/0697; C12N 5/0658; C12N 5/0656; C12M 21/08; C12M 23/12; C12M 41/46; C12M 35/02; G01N 33/5088; G01N 33/5061; C12M 35/08; C12N 2533/54; C12N 2513/00; C12N 2533/56

(57) ABSTRACT

Embodiments described herein relate generally to a three-dimensional ex vivo skeletal muscle tissue comprising a hydrogel and a plurality of cells that includes skeletal muscle cells, at least a portion of the cells being encapsulated inside the hydrogel. In some embodiments, the skeletal muscle tissue is characterized by one or more contractions in response to an electrical and/or chemical stimulation.

### RELATED APPLICATIONS

This application claims priority to and the benefit of U.S. Provisional Patent Application No. 62/940,976, filed on Nov. 27, 2019, the disclosure of which is hereby incorporated by reference in its entirety.

### TECHNICAL FIELD

The present disclosure relates generally to artificial skeletal muscle tissues and use thereof.

### BACKGROUND

As one of three major muscle types, skeletal muscle is a form of striated muscle tissue, which is under the voluntary control of the somatic nervous system. Most skeletal muscles are attached to bones by bundles of collagen fibers known as tendons.

Two-dimensional in vitro skeletal muscle tissues have been made, but they do not fully recapitulate the organization and function of native skeletal muscle, limiting their use in physiological and pharmacological studies.

### SUMMARY

Embodiments described herein relate generally to three-dimensional (3D) artificial skeletal muscle tissues, methods of producing the tissues, and methods of using the tissues.

One aspect of the present disclosure relates to a 3D ex vivo skeletal muscle tissue comprising a hydrogel and a plurality of cells that includes skeletal muscle cells, wherein at least a portion of the cells are encapsulated inside the hydrogel, and wherein the skeletal muscle tissue is characterized by one or more contractions in response to an electrical and/or chemical stimulation.

In some embodiments, the plurality of cells further comprises fibroblasts.

In some embodiments, the fibroblasts and the skeletal muscle cells are at a ratio of between about 1:5 and 1:50.

In some embodiments, the skeletal muscle cells comprise human skeletal muscle cells.

In some embodiments, the hydrogel comprises collagen or a collagen derivative, intestinal submucosa or a derivative thereof, cellulose or a cellulose derivative, a proteoglycan, heparin sulfate, chondroitin sulfate, keratin sulfate, hyaluronic acid, elastin, fibronectin, laminin, fibrin, chitosan, alginate, Matrigel®, Geltrex, agarose, decellularized extracellular matrix, polyethylene glycol or a derivative thereof, silicone or a derivative thereof, or a combination thereof. In some embodiments, the hydrogel comprises Matrigel®.

In some embodiments, the collagen comprises Type I collagen, Type III collagen, Type IV collagen, Type V collagen, Type XI collagen, Type XII collagen, or a combination thereof.

In some embodiments, at least 50% of the cells are encapsulated inside the hydrogel.

In some embodiments, the 3D ex vivo skeletal muscle tissue further comprises fibrinogen and/or thrombin.

In some embodiments, the skeletal muscle tissue has about 30,000 to about 1,000,000 cells.

In some embodiments, the skeletal muscle tissue has a volume of about 0.1 mm³ to about 2.5 mm³.

In some embodiments, the skeletal muscle tissue comprises an A band, an I band, a Z line, a M line, a H zone, or a combination thereof.

In some embodiments, the skeletal muscle tissue comprises cross-striations, elongated nuclei, or a combination thereof.

In some embodiments, the skeletal muscle tissue comprises an acetylcholine receptor, slow twitch fibers, fast twitch fibers, or a combination thereof.

In some embodiments, expression levels of genes related to maturation, genes related to calcium handling, and/or genes related to sarcomeric proteins are substantially the same as those in a native human skeletal muscle tissue.

In some embodiments, the one or more contractions generate a twitch force and/or a tetanic force.

In some embodiments, the chemical stimulation comprises acetylcholine, adenosine triphosphate, 4-chloro-m-cresol, or a combination.

In some embodiments, the skeletal muscle tissue is characterized by a transient change in intracellular calcium concentration in response to an electrical and/or chemical stimulation. In some embodiments, the skeletal muscle tissue is characterized by a shortened action potential with prominent hyperpolarization.

One aspect of the present disclosure relates to a tissue system comprising a 3D ex vivo skeletal muscle tissue described herein and a bioreactor, wherein the bioreactor comprises: a device having a well configured for growing the 3D ex vivo skeletal muscle tissue from the cells seeded therein, wherein the well has a bottom; and at least two elastic sensing elements disposed across the well such that there is a gap between the sensing elements and the bottom of the well, wherein the sensing elements are configured to: (a) permit attachment of the 3D ex vivo skeletal muscle tissue formed therebetween, thereby suspending the 3D ex vivo skeletal muscle tissue above the bottom of the well, and (b) deform in response to a contractile force exerted on the sensing elements by the three-dimensional ex vivo skeletal muscle tissue.

In some embodiments, the bioreactor further comprises at least two electrodes configured to apply an electrical stimulation to the 3D ex vivo skeletal muscle tissue of the bioreactor.

In some embodiments, the sensing elements comprise a synthetic polymer, a biologic polymer, or a combination thereof.

In some embodiments, the polymer is degradable.

In some embodiments, the polymer is nondegradable.

In some embodiments, the at least two elastic sensing elements comprise a polymer selected from the group consisting of polylactic acid, poly(lactic-co-glycolic) acid, poly(caprolactone), polyglycolide, polylactide, polyhydroxobutyrate, polyhydroxyalcanoic acid, chitosan, hyaluronic acid, poly(2-hydroxyethyl-methacrylate), poly(ethylene glycol), poly(L-lactide) (PLA), poly(dimethysiloxane) (PDMS), poly(methylmethacrylate) (PMMA), poly(glycerol sebacate), poly(octamethylene maleate (anhydride) citrate) (POMaC), POMaC without citric acid, poly(ε-caprolactone), polyurethane, silk, and a combination thereof.

In some embodiments, the polymer comprises POMaC.

In some embodiments, the sensing elements have an elasticity from about 10 kPa to 0.8 MPa.

In some embodiments, the sensing elements are in the form of polymer wires.

In some embodiments, the bioreactor comprises 2 to 25 sensing elements per well.

In some embodiments, the bioreactor comprises a multi-well plate.

In some embodiments, the multi-well plate comprises 6 wells, 8 wells, 12 wells, 24 wells, 96 wells, 384 wells, or 1536 wells.

One aspect of the present disclosure relates to a method for measuring an effect of a test agent on contraction using the tissue system described herein, comprising: measuring a first value of a contraction characteristic of the 3D ex vivo skeletal muscle tissue in the bioreactor before exposure to the test agent; contacting the 3D ex vivo skeletal muscle tissue with the test agent under conditions sufficient for the test agent to modulate the contraction; measuring a second value of the contraction characteristic of the 3D ex vivo skeletal muscle tissue after exposure to the test agent; and determining whether the test agent modulates the contraction by comparing the first value with the second value.

In some embodiments, the test agent modulates the contraction when there is a significant difference between the first value and the second value.

In some embodiments, the test agent is selected from the group consisting of a small molecule, an antibody, an ion, a protein, a peptide, a lipid, DNA, RNA, a virus, bacteria, a microparticle, a nanoparticle, a therapeutic agent, and a toxin.

One aspect of the present disclosure relates to a method for measuring an effect of a test agent on a calcium transient using the tissue system described herein, comprising: measuring a first value of a calcium transient characteristic of the 3D ex vivo skeletal muscle tissue in the bioreactor before exposure to the test agent; contacting the 3D ex vivo skeletal muscle tissue with the test agent under conditions sufficient for the test agent to modulate the calcium transient; measuring a second value of the calcium transient characteristic of the 3D ex vivo skeletal muscle tissue after exposure to the test agent; and determining whether the test agent modulates the calcium transient by comparing the first value with the second value.

In some embodiments, measuring the first value or second value comprises measuring a fluorescence signal of an intracellular calcium indicator in the 3D ex vivo skeletal muscle tissue.

In some embodiments, the intracellular calcium indicator is selected from Fura-4F AM, Fura-2, Fluo-3, Fluo-4, and Indo-1, Mag-Fura-5, and Mag-Fura-red.

In some embodiments, the test agent modulates the calcium transient when there is a significant difference between the first calcium transient and the second calcium transient.

In some embodiments, the test agent is selected from the group consisting of a small molecule, an antibody, an ion, a protein, a peptide, a lipid, DNA, RNA, a virus, bacteria, a microparticle, a nanoparticle, a therapeutic agent, and a toxin.

### BRIEF DESCRIPTION OF THE DRAWINGS

FIG. 1 is a set of optical images showing a representative in vitro human skeletal tissue, pictured in the Biowire™ II platform, composed of PromoCell skeletal muscle cells (SkMCs) with or without cardiac fibroblasts (cFBs). The presence of fibroblasts did not significantly affect tissue compaction. The optical images were acquired 12 days after cell seeding.

FIG. 2 is a series of representative contractility traces showing the active force generated by skeletal tissues. After 4 days of continuous electrical stimulation, skeletal tissues were not spontaneously beating (SPT) and when electrically stimulated were able to capture at increasing frequencies (1 Hz, 2 Hz).

FIG. 3 is a series of representative contractility traces showing the active force generated overtime by the skeletal tissues.

FIGS. 4A-4C are schematic diagrams showing the stimulation protocol used for maturation of skeletal tissue using (FIG. 4A) low frequency continuous stimulation, (FIG. 4B) high frequency burst stimulation with fixed burst frequency, and (FIG. 4C) high frequency burst stimulation with increasing frequency of burst stimulation.

### DETAILED DESCRIPTION

One aspect of the present disclosure relates to a 3D ex vivo skeletal muscle tissue comprising a hydrogel and a plurality of cells that includes skeletal muscle cells. In some embodiments, at least a portion of the cells are encapsulated inside the hydrogel, and the skeletal muscle tissue is characterized by one or more contractions in response to an electrical and/or chemical stimulation.

In some embodiments, the electrical stimulation to elicit the one or more contractions can be about 0.1-100 Hz.

In some embodiments, the chemical stimulation to elicit the one or more contractions comprises acetylcholine, adenosine triphosphate, 4-Chloro-m-cresol, or a combination thereof.

In some embodiments, the plurality of cells can further include fibroblasts. In some embodiments, the number of fibroblasts and the number of skeletal muscle cells can be at a ratio of no more than about 1:3, no more than about 1:3.5, no more than about 1:4, no more than about 1:4.5, no more than about 1:5, no more than about 1:5.5, or no more than about 1:6. In some embodiments, the number of fibroblasts and the number of skeletal muscle cells can be at a ratio of at least about 1:70, at least about 1:65, at least about 1:60, at least about 1:55, at least about 1:50, at least about 1:45, or at least about 1:40.

Combinations of the above-referenced ranges for the ratio of the fibroblasts over the skeletal muscle cells are also possible (e.g., at least about 1:70 to no more than about 1:3, or at least about 1:60 to no more than about 1:4.5), inclusive of all values and ranges therebetween. For example, the ratio of the fibroblasts over the skeletal muscle cells can be between about 1:50 and 1:5.

In some embodiments, the plurality of cells can further include myoblasts. The plurality of cells can further include other cell types, including endothelial cells, immune cells, and neurons.

The cells can be derived from any animal including human or non-human animals. In some embodiments, the cells are human cells. In some embodiments, the cells are non-human cells, such as rat cells and mouse cells. Accordingly, the skeletal muscle cells can be derived from a human and/or a non-human animal; the fibroblasts can be derived from a human and/or a non-human animal; the myoblasts can be derived from a human and/or a non-human animal; the endothelial cells can be derived from a human and/or a non-human animal; and the immune cells can be derived from a human and/or a non-human animal.

Depending on the animal from which the cells are derived, the skeletal muscle tissue produced therefrom can exhibit the same or substantially the same characteristics as a skeletal muscle tissue of the animal, such as a human skeletal muscle tissue, a mouse skeletal muscle tissue, or a rat skeletal muscle tissue.

The hydrogel can include collagen or a collagen derivative, intestinal submucosa or a derivative thereof, cellulose or a cellulose derivative, a proteoglycan, heparin sulfate, chondroitin sulfate, keratin sulfate, hyaluronic acid, elastin, fibronectin, thrombin, laminin, fibrin, chitosan, alginate, Matrigel®, Geltrex, agarose, decellularized extracellular matrix, polyethylene glycol or a derivative thereof, silicone or a derivative thereof, or a combination thereof. In some embodiments, the hydrogel can include Matrigel® or Geltrex.

In some embodiments, the hydrogel can include at least about 1 wt % collagen or a collagen derivative, at least about 2 wt % collagen or a collagen derivative, at least about 3 wt % collagen or a collagen derivative, at least about 4 wt % collagen or a collagen derivative, at least about 5 wt % collagen or a collagen derivative, at least about 6 wt % collagen or a collagen derivative, at least about 7 wt % collagen or a collagen derivative, at least about 8 wt % collagen or a collagen derivative, at least about 9 wt % collagen or a collagen derivative, or at least about 10 wt % collagen or a collagen derivative.

In some embodiments, the hydrogel can include no more than about 50 wt % collagen or a collagen derivative, no more than about 45 wt % collagen or a collagen derivative, no more than about 40 wt % collagen or a collagen derivative, no more than about 35 wt % collagen or a collagen derivative, no more than about 30 wt % collagen or a collagen derivative, no more than about 25 wt % collagen or a collagen derivative, no more than about 20 wt % collagen or a collagen derivative, or no more than about 15 wt % collagen or a collagen derivative.

Combinations of the above-referenced ranges for the weight ratio of collagen or a collagen derivative in the hydrogel are also possible (e.g., at least about 1 wt % to no more than about 50 wt %, or at least about 5 wt % to no more than about 40 wt %), inclusive of all values and ranges therebetween.

In some embodiments, the collagen comprises Type I collagen, Type III collagen, Type IV collagen, Type V collagen, Type XI collagen, Type XII collagen, or a combination thereof.

In some embodiments, at least about 40% of the cells are encapsulated inside the hydrogel. In some embodiments, at least about 45% of the cells are encapsulated inside the hydrogel. In some embodiments, at least about 50% of the cells are encapsulated inside the hydrogel. In some embodiments, at least about 55% of the cells are encapsulated inside the hydrogel. In some embodiments, at least about 60% of the cells are encapsulated inside the hydrogel. In some embodiments, at least about 65% of the cells are encapsulated inside the hydrogel. In some embodiments, at least about 70% of the cells are encapsulated inside the hydrogel.

In some embodiments, about 100% of the cells are encapsulated inside the hydrogel. In some embodiments, no more than about 99% of the cells are encapsulated inside the hydrogel. In some embodiments, no more than about 95% of the cells are encapsulated inside the hydrogel. In some embodiments, no more than about 90% of the cells are encapsulated inside the hydrogel. In some embodiments, no more than about 85% of the cells are encapsulated inside the hydrogel. In some embodiments, no more than about 80% of the cells are encapsulated inside the hydrogel.

Combinations of the above-referenced ranges for the percentage of cells encapsulated in the hydrogel are also possible (e.g., at least about 40% to about 100%, or at least about 50% to no more than about 99%), inclusive of all values and ranges therebetween.

In some embodiments, the 3D ex vivo skeletal muscle tissue can further include fibrinogen and/or thrombin. The fibrinogen can include human and/or non-human fibrinogen. In some embodiments, the thrombin can include human and/or non-human thrombin.

In some embodiments, the volume of the 3D ex vivo skeletal muscle tissue can be at least about 0.1 mm³, at least about 0.5 mm³, at least about 1 mm³, at least about 1.5 mm³, at least about 2 mm³, or at least about 2.5 mm³.

In some embodiments, the volume of the 3D ex vivo skeletal muscle tissue can be no more than about 10 mm³, no more than about 9 mm³, no more than about 8 mm³, no more than about 7 mm³, no more than about 6 mm³, no more than about 5 mm³, no more than about 4 mm³, no more than about 3 mm³, or no more than about 2.5 mm³.

Combinations of the above-referenced ranges for the volume of the 3D ex vivo skeletal muscle tissue are also possible (e.g., at least about 0.1 mm³ to no more than about 10 mm³, or at least about 0.1 mm³ to no more than about 2.5 mm³), inclusive of all values and ranges therebetween.

The number of cells in the 3D ex vivo skeletal muscle tissue depends on the volume of the tissue. In some embodiments, the number of cells can be at least about 20,000, at least about 25,000, at least about 30,000, at least about 35,000, at least about 40,000, at least about 45,000, at least about 50,000. In some embodiments, the number of cells can be no more than about 5,000,000, no more than about 2,000,000, no more than about 1,000,000, no more than about 900,000, no more than 800,000, no more than about 700,000, no more than 600,000, no more than about 500,000, no more than 400,000, no more than about 300,000, no more than 200,000, or no more than 100,000.

Combinations of the above-referenced ranges for the number of cells in the tissue are also possible (e.g., at least about 20,000 to no more than about 5,000,000, or at least about 30,000 to no more than about 1,000,000), inclusive of all values and ranges therebetween.

The 3D ex vivo skeletal muscle tissue can be characterized by morphology, structural organization, elasticity, extensibility, gene expression, protein expression, excitability, contractility, calcium transients, electrophysiology, biochemical signaling, or a combination thereof. The 3D ex vivo skeletal muscle tissue can have one or more features substantially the same as a healthy native human skeletal muscle tissue. In some embodiments, the 3D ex vivo skeletal muscle tissue can fully recapitulate the organization and function of native skeletal muscle tissues, e.g., human skeletal muscle tissues.

In some embodiments, the 3D ex vivo skeletal muscle tissue can include an A band, an I band, a Z line, a M line, a H zone, a T tubule, or a combination thereof.

In some embodiments, the 3D ex vivo skeletal muscle tissue can include cross-striations, elongated nuclei, or a combination thereof. In some embodiments, the 3D ex vivo skeletal muscle tissue can include an acetylcholine receptor, slow twitch fibers, fast twitch fibers, or a combination thereof. Slow twitch fibers and fast twitch fibers in skeletal muscle tissues are disclosed for example in C. Handschin, et al., “External Physical and Biochemical Stimulation to Enhance Skeletal Muscle Bioengineering,” Adv. Drug Deliv. Rev. 2015, 82-83, 168-175; D. Pette and G. Vrbova, “The Contribution of Neuromuscular Stimulation in Elucidating Muscle Plasticity Revisited,” Eur. J. Transl. Myol. 2017, 27, 33-39, the contents of each of which are incorporated herein by reference.

In some embodiments, expression levels of genes related to maturation (e.g., myosin isoforms ratio), genes related to calcium handling, and/or genes related to sarcomeric proteins (e.g., dystrophin, myosin heavy chain, or alpha actinin) in the 3D ex vivo skeletal muscle tissue are substantially the same as those in a native human skeletal muscle tissue.

In some embodiments, the one or more contractions generate a twitch force and/or a tetanic force.

In some embodiments, the 3D ex vivo skeletal muscle tissue can be characterized by a transient change in intracellular calcium concentration in response to an electrical and/or chemical stimulation. The transient changes in intracellular calcium concentration can be a consequence of sarcolemmal depolarization, resulting in myofiber contractility. In some embodiments, the electrical stimulation can be about 0.1-100 Hz. In some embodiments, the chemical stimulation comprises acetylcholine, adenosine triphosphate, 4-chloro-m-cresol, or a combination thereof.

In some embodiments, the electrophysiology of the 3D ex vivo skeletal muscle tissue can be characterized by a shortened action potential with prominent hyperpolarization.

One aspect of the present disclosure relates to a 3D ex vivo diseased skeletal muscle tissue that includes a plurality of diseased skeletal muscle cells. As compared to healthy skeletal muscle tissues, the 3D ex vivo disease skeletal muscle tissue can be characterized by reduced force generation-capacity, alteration in calcium handling, alteration in electrophysiology, reduced proliferation and differentiation ability, alteration in extracellular matrix material (ECM), or a combination thereof. In some embodiments, when the 3D ex vivo diseased skeletal muscle tissue is excited by an electrical and/or chemical stimulation, the tissue does not contract or contracts at a significantly reduced amount as compared to a healthy skeletal muscle tissue.

Muscle cells adhere to and connect with the ECM. In addition, the ECM can provide an appropriate and permissive environment for muscle development and functioning. Alternations in the ECM can signal a disease state in the skeletal muscle tissue. See K. Grzelkowska-Kowalczyk, “The Importance of Extracellular Matrix in Skeletal Muscle Development and Function” in “Composition and Function of the Extracellular Matrix in the Human Body,” edited by F. Travascio, IntechOpen 2016, the contents of which are incorporated herein by reference.

In some embodiments, tissues of interest can be treated with agents known in the art to cause cellular damage (e.g., toxins, mutagens, radiation, infectious agents, or chemical agents), inducing injury in the tissue. In some embodiments, tissues of interest can be altered using standard recombinant techniques to induce a disease state. For example, techniques of homologous recombination can be used to insert a transgene into a cell, or “knock-out” gene expression of a gene of interest. For a review of homologous recombination, see Lewin, B., Genes V, Oxford University Press, New York, 1994, pp. 968-997; and Capecchi, M., (1989) Science 244:1288-1292; Capecchi, M., (1989) Trends Genet. 5 (3):70-76. In some embodiments, the tissue of interest is injured as a result of an inherited genetic defect, which can be a single gene defect or a multifactorial defect.

The 3D ex vivo diseased skeletal muscle tissue can be used in a disease model. For example, the disease model can mimic one or more muscle diseases such as Duchenne muscular dystrophy, Facioscapulohumeral muscular dystrophy, myotonic dystrophy, congenital fiber type disproportion, myosinopathies, Pompe disease, obesity and type 2 diabetes, muscle inactivity, aging/sarcopenia, heart failure, and chronic obstructive pulmonary disease. See J. Talbot and L. Mayes, “Skeletal muscle fiber type: using insights from muscle developmental biology to dissect targets for susceptibility and resistance to muscle disease,” Wiley Interdiscip Rev Dev Biol. 2016, 5, 518-534, the contents of which are incorporated herein by reference.

One aspect of the present disclosure relates to a tissue system comprising a 3D ex vivo skeletal muscle tissue described herein and a bioreactor. In some embodiments, the bioreactor comprises: a device having a well configured for growing the 3D ex vivo skeletal muscle tissue from the cells seeded therein, wherein the well has a bottom; and at least two elastic sensing elements disposed across the well such that there is a gap between the sensing elements and the bottom of the well. There is also a gap between the bottom of the well and the tissue suspended on the at least two sensing elements.

The shape of the well is not limited in any particular manner and can be square, rectangular, circular, oval, oblong, triangular, or any combination of shapes. The other dimensions of the well also may vary in any suitable manner. For example, the depth of the well, height of the well, and length of the well, and the overall volume of the well may be varied in any suitable way.

For example, the length, height, or width of the well can be about 0.1-1 mm, about 0.2-2 mm, about 0.3-3 mm, about 0.4-4 mm, about 0.5-5 mm, about 0.6-6 mm, about 0.7-7 mm, about 0.8-8 mm, about 0.9-9 mm, about 1-10 mm, about 1-100 mm, or about 10-100 mm.

The well can be characterized by a longitudinal axis. The longitudinal axis can be along the length of the well.

In some embodiments, the well can be positioned inside a cell culture well on a multi-well plate. In some embodiments, the multi-well plate can include a plurality of wells, such as 6 wells, 8 wells, 12 wells, 24 wells, 48 wells, 96 wells, 384 wells, or 1536 wells.

The at least two sensing elements can function as anchor points for a tissue formed therebetween. The sensing elements can be configured to: (a) permit attachment of the 3D ex vivo skeletal muscle tissue formed therebetween, thereby suspending the 3D ex vivo skeletal muscle tissue above the bottom of the well, and/or (b) deform in response to a contractile force exerted on the sensing elements by the 3D ex vivo skeletal muscle tissue.

The bioreactor is not limited to having two such sensing elements per well, but may include more than two, such as 2-30 sensing elements per well, e.g., 2-25, 2-20, 2-15, or 2-10 sensing elements per well. In some embodiments, the bioreactor can include 2, 3, 4, 5, 6, 7, 8, 9, or more sensing elements per well. Any number of sensing elements per well may be provided so long as there is the ability to form a tissue that forms around each of the sensing elements and becomes joined therebetween such that the tissue is suspended above the bottom of the well.

The sensing elements can have an orientation that is perpendicular or substantially perpendicular to the longitudinal axis of the well. The tissue can be aligned in the same or substantially the same direction as the longitudinal axis of the well.

The sensing elements can have an orientation that is parallel or substantially parallel to the longitudinal axis of the well.

The sensing elements can have an orientation that is diagonal or substantially diagonal to the longitudinal axis of the well.

The sensing elements can include a polymer. The polymer can be synthetic or biologic. The polymer can also be biodegradable or non-biodegradable.

In some embodiments, the sensing elements can comprise a polymer having a Young's modulus in the range of 10 kPa to 800 kPa. For example, the polymer can have a Young's modulus in the range of 20 kPa to 700 kPa, 20 kPa to 600 kPa, 20 kPa to 500 kPa, 50 kPa to 500 kPa, or 100 kPa to 500 kPa. In some embodiments, the polymer can have a Young's modulus of about 150 kPa, about 200 kPa, about 250 kPa, about 300 kPa, about 350 kPa, about 400 kPa, about 450 kPa, about 500 kPa, or about 550 kPa.

In some embodiments, the sensing elements can comprise a polymer whose mechanical properties are tunable by controlling the polymerization using different crosslinking energy. Tunability can also be controlled by the ratio of the mixtures of polymer units during the polymerization reaction.

In some embodiments, the polymer can be at least one of polylactic acid, poly(lactic-co-glycolic) acid, poly(caprolactone), polyglycolide, polylactide, polyhydroxobutyrate, polyhydroxyalcanoic acid, chitosan, hyaluronic acid, a hydrogel, poly(2-hydroxyethyl-methacrylate), poly(ethylene glycol), poly(L-lactide) (PLA), poly(dimethysiloxane) (PDMS), poly(methylmethacrylate) (PMMA), poly(glycerol sebacate), poly(octamethylene maleate (anhydride) citrate) (POMaC), POMaC without citric acid, poly(ε-caprolactone), polyurethane, silk, a nanofabricated material, a co-polymer, a blended polymer, or a combination thereof. In some embodiments, the sensing elements comprise POMaC.

The shape, thickness, length, orientation, and surface topographical properties of the sensing elements can vary any number of suitable ways so long as the sensing elements are capable of deforming, bending, or otherwise changing shape in response to the contractile action or activity of the tissue connected therebetween, and that such deforming, bending, or otherwise shape changing can be reliably measured. In some embodiments, the sensing elements are in the form of wires, e.g., polymer wires.

In some embodiments, the sensing elements are porous, thereby permitting delivery of nutrients and growth factors to the skeletal muscle tissue.

To produce the skeletal muscle tissue, the cells can be seeded in a hydrogel. The hydrogel can include collagen or a collagen derivative, intestinal submucosa or a derivative thereof, cellulose or a cellulose derivative, a proteoglycan, heparin sulfate, chondroitin sulfate, keratin sulfate, hyaluronic acid, elastin, fibronectin, laminin, fibrin, chitosan, alginate, Matrigel®, Geltrex, agarose, decellularized extracellular matrix, polyethylene glycol or a derivative thereof, silicone or a derivative thereof, or a combination thereof. In some embodiments, the hydrogel comprises Matrigel® or Geltrex.

Neurons can be seeded in a location in the bioreactor different from the skeletal muscle tissue. The neurons can then grow and/or extend via a channel to contact the skeletal muscle tissue.

In some embodiments, the number of neurons and the number of skeletal muscle cells can be at a ratio of no more than about 1:3, no more than about 1:3.5, no more than about 1:4, no more than about 1:4.5, no more than about 1:5, no more than about 1:5.5, or no more than about 1:6. In some embodiments, the number of neurons and the number of skeletal muscle cells can be at a ratio of at least about 1:70, at least about 1:65, at least about 1:60, at least about 1:55, at least about 1:50, at least about 1:45, or at least about 1:40.

Combinations of the above-referenced ranges for the ratio of the neurons over the skeletal muscle cells are also possible (e.g., at least about 1:70 to less than about 1:3, at least about 1:60 to less than about 1:4.5), inclusive of all values and ranges therebetween. For example, the ratio of the neurons over the skeletal muscle cells can be between about 1:50 and 1:5.

The bioreactors can further include electrodes configured to provide electrical stimulations to the skeletal muscle tissue. Each electrode can include conductive carbon, gold, platinum, palladium, stainless steel, tin, tungsten, titanium, or a combination thereof. Further examples of conductive materials for tissue stimulation can be found at Merrill et al., “Electrical Stimulation of Excitable Tissue: Design of Efficacious and Safe Protocols,” J. of Neuroscience Methods 2005, 141, 171-198; Tandon et al., “Characterization of Electrical Stimulation Electrodes for Cardiac Tissue Engineering,” Proceedings of the 28^{th} IEEE, pages 845-848, the contents of each of which are incorporated herein by reference.

Additional features of the bioreactor can be found at US20160282338, the contents of which are incorporated by reference in their entireties.

In general, to produce mature 3D ex vivo skeletal muscle tissues, the cells embedded in the hydrogel are subject to electrical stimulations in accordance with a stimulation protocol.

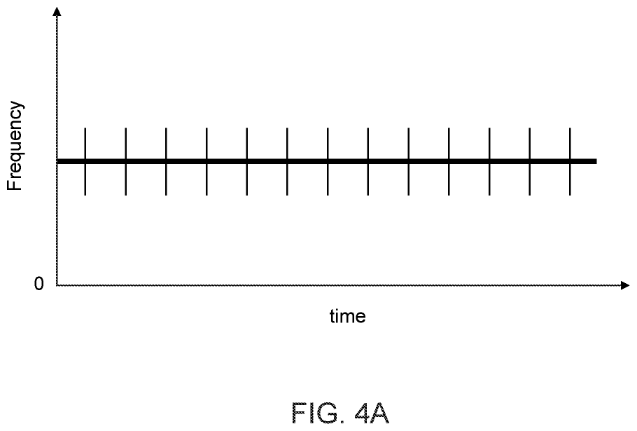

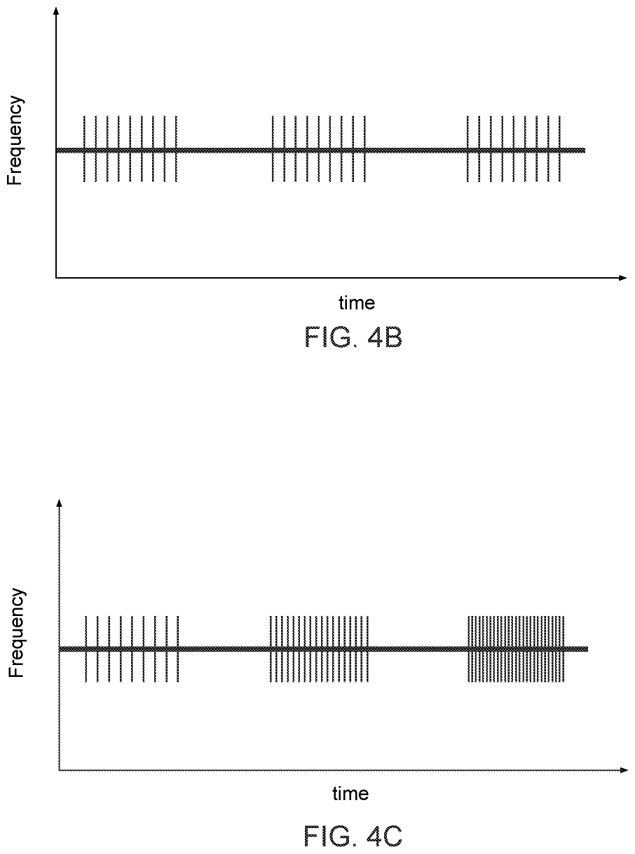

In one aspect, the stimulation protocol can include a low frequency for an extended duration. See FIG. 4A, which is a schematic diagram illustrating an example of such stimulation protocol.

In some embodiments, the low frequency can be at least about 0.1 Hz, at least about 0.2 Hz, at least about 0.3 Hz, at least about 0.4 Hz, at least about 0.5 Hz, or at least about 0.6 Hz, at least about 0.7 Hz, at least about 0.8 Hz, or at least about 0.9 Hz, at least about 1 Hz, at least about 1.5 Hz, at least about 2 Hz, at least about 2.5 Hz, or at least about 3 Hz. In some embodiments, the low frequency can be no more than about 5 Hz, no more than about 4.5 Hz, no more than about 4 Hz, no more than about 3.5 Hz, no more than about 3 Hz, no more than about 2.5 Hz, no more than about 2 Hz, no more than about 1.5 Hz, or no more than about 1 Hz.

Combinations of the above-referenced ranges for the low frequency are also possible (e.g., at least about 0.1 Hz to no more than about 1 Hz, or at least about 0.5 Hz to no more than about 2 Hz), inclusive of all values and ranges therebetween. In some embodiments, the low frequency is about 0.1 Hz, about 0.2 Hz, about 0.3 Hz, about 0.4 Hz, about 0.5 Hz, about 0.6 Hz, about 0.7 Hz, about 0.8 Hz, about 0.9 Hz, or about 1.0 Hz.

In some embodiments, the extended duration can be at least about 1 hour, at least about 2 hours, at least about 3 hours, at least about 4 hours, at least about 5 hours, at least about 10 hours, at least about 20 hours, at least about 40 hours, or at least about one week. In some embodiments, the extended duration can be no more than about 3 weeks, no more than about 2.5 weeks, no more than about 2 weeks, no more than about 1.5 weeks, or more than about one week, no more than about 3 days, or no more than about 24 hours.

Combinations of the above-referenced ranges for the extended duration are also possible (e.g., at least about 1 hour to no more than about 3 weeks, or at least about 5 hours to no more than about 2 weeks), inclusive of all values and ranges therebetween.

In another aspect, the stimulation protocol can include a plurality of high frequency pulses separated by no electrical stimulations. As compared to mature 3D ex vivo cardiac tissues, high frequency pulses (e.g., at least about 10 Hz) may be required to produce mature 3D ex vivo skeletal muscle tissues. Without wishing to be bound by theory, the high frequency requirement is because of the ability of skeletal tissue to generate sustained contractures under repeat high frequency pulses. Repeat sustained contractures results in maturation of skeletal tissue. See FIG. 4B, which is a schematic diagram illustrating an example of such stimulation protocol.

In some embodiments, the high frequency can be at least about 10 Hz, at least about 15 Hz, at least about 20 Hz, at least about 25 Hz, at least about 30 Hz, at least about 35 Hz, or at least about 40 Hz. In some embodiments, the high frequency can be no more than about 100 Hz, no more than about 95 Hz, no more than about 90 Hz, no more than about 85 Hz, no more than about 80 Hz, no more than about 75 Hz, no more than about 70 Hz, no more than about 65 Hz, or no more than about 60 Hz.

Combinations of the above-referenced ranges for the high frequency are also possible (e.g., at least about 10 Hz to no more than about 100 Hz, or at least about 10 Hz to no more than about 60 Hz), inclusive of all values and ranges therebetween.

In some embodiments, the duration for each pulse can be at least about one second, at least about 10 seconds, at least about 30 seconds, at least about 60 seconds, at least about 5 minutes, at least about 10 minutes, at least about 15 minutes, at least about 20 minutes, at least about 25 minutes, at least about 30 minutes, at least about 35 minutes, at least about 40 minutes, at least about 45 minutes, at least about 50 minutes, at least about 55 minutes, at least about 60 minutes. In some embodiments, the duration for each pulse can be no more than about 3 hours, no more than about 2.5 hours, no more than about 2 hours, no more than about 1.5 hours, or no more than about 1 hour.

Combinations of the above-referenced ranges for the duration for each pulse are also possible (e.g., at least about one second to no more than about 3 hours, or at least about 30 seconds to no more than about 1 hour), inclusive of all values and ranges therebetween.

In some embodiments, the stimulation protocol can include about 1-60 high frequency pulses per hour, e.g., about 1-55, about 1-50, about 1-45, about 1-40, or about 10-60 high frequency pulses per hour.

In yet another aspect, the stimulation protocol can include a first frequency for a first duration, a second frequency for a second duration, wherein the first frequency is increased to the second frequency at a first ramp rate. See FIG. 4C, which is a schematic diagram illustrating an example of such stimulation protocol.

In some embodiments, the first frequency can be at least about 0.1 Hz, at least about 0.2 Hz, at least about 0.3 Hz, at least about 0.4 Hz, at least about 0.5 Hz, or at least about 0.6 Hz, at least about 0.7 Hz, at least about 0.8 Hz, or at least about 0.9 Hz, at least about 1 Hz, at least about 1.5 Hz, at least about 2 Hz, at least about 2.5 Hz, or at least about 3 Hz. In some embodiments, the first frequency can be no more than about 5 Hz, no more than about 4.5 Hz, no more than about 4 Hz, no more than about 3.5 Hz, no more than about 3 Hz, no more than about 2.5 Hz, no more than about 2 Hz, no more than about 1.5 Hz, or no more than about 1 Hz.

Combinations of the above-referenced ranges for the first frequency are also possible (e.g., at least about 0.1 Hz to no more than about 1 Hz, or at least about 0.5 Hz to no more than about 2 Hz), inclusive of all values and ranges therebetween. In some embodiments, the first frequency is about 0.1 Hz, about 0.2 Hz, about 0.3 Hz, about 0.4 Hz, about 0.5 Hz, about 0.6 Hz, about 0.7 Hz, about 0.8 Hz, about 0.9 Hz, or about 1.0 Hz.

In some embodiments, the first duration can be at least about one second, at least about 10 seconds, at least about 30 seconds, at least about 60 seconds, at least about 5 minutes, at least about 10 minutes, at least about 15 minutes, at least about 20 minutes, at least about 25 minutes, at least about 30 minutes, at least about 35 minutes, at least about 40 minutes, at least about 45 minutes, at least about 50 minutes, at least about 55 minutes, at least about 60 minutes. In some embodiments, the first duration can be no more than about 3 hours, no more than about 2.5 hours, no more than about 2 hours, no more than about 1.5 hours, or no more than about 1 hour.

Combinations of the above-referenced ranges for the first duration are also possible (e.g., at least about one second to no more than about 3 hours, or at least about 30 seconds to no more than about 1 hour), inclusive of all values and ranges therebetween. In some embodiments, the first duration is about 1 hour.

In some embodiments, the second frequency can be at least about 10 Hz, at least about 15 Hz, at least about 20 Hz, at least about 25 Hz, at least about 30 Hz, at least about 35 Hz, or at least about 40 Hz. In some embodiments, the second frequency can be no more than about 100 Hz, no more than about 95 Hz, no more than about 90 Hz, no more than about 85 Hz, no more than about 80 Hz, no more than about 75 Hz, no more than about 70 Hz, no more than about 65 Hz, or no more than about 60 Hz.

Combinations of the above-referenced ranges for the second frequency are also possible (e.g., at least about 10 Hz to no more than about 100 Hz, or at least about 10 Hz to no more than about 60 Hz), inclusive of all values and ranges therebetween.

In some embodiments, the second duration can be at least about one second, at least about 10 seconds, at least about 30 seconds, at least about 60 seconds, at least about 5 minutes, at least about 10 minutes, at least about 15 minutes, at least about 20 minutes, at least about 25 minutes, at least about 30 minutes, at least about 35 minutes, at least about 40 minutes, at least about 45 minutes, at least about 50 minutes, at least about 55 minutes, at least about 60 minutes. In some embodiments, the second duration can be no more than about 3 hours, no more than about 2.5 hours, no more than about 2 hours, no more than about 1.5 hours, or no more than about 1 hour.

Combinations of the above-referenced ranges for the second duration are also possible (e.g., at least about one second to no more than about 3 hours, or at least about 30 seconds to no more than about 1 hour), inclusive of all values and ranges therebetween. In some embodiments, the second duration is about 1 hour.

In some embodiments, the first ramp rate can be at least about 0.1 Hz/hour.

In some embodiments, the stimulation protocol can further include a third frequency for a third duration. The third frequency can be either smaller or greater than the second frequency. The stimulation protocol can further include a fourth frequency for a fourth duration, a fifth frequency for a fifth duration, etc.

Any one of the above stimulation protocols can be applied to the cells once a day, twice a day, three times a day, or more than three times a day.

In some embodiments, the electrical stimulation is terminated when a certain phenotype is achieved, e.g., slow twitch fibers or fast twitch fibers, which can be analyzed based on the expression of markers (type II myosin heavy chain mainly expressed in fast twitch fibers and type I myosin heavy chain mainly expressed in slow twitch fibers). Parameters of contractility and calcium transients can also allow the characterization of slow twitch fibers and fast twitch fibers.

In some embodiments, the electrical stimulation is terminated when one or more measurable parameters are achieved, e.g., a minimum tetanus to baseline ratio of greater than 2, or a minimum tetanus force of 50 μN.

In some embodiments, the tissue system described herein can be used for measuring the effect on contractility of the skeletal muscle tissue formed therein resulting from exposure to a test agent of interest.

In some embodiments, the tissue system can be used for (a) testing of the efficacy and safety (including toxicity) of a test agent (e.g., an experimental pharmacologic agent), (b) defining the pharmacokinetics and/or pharmacodynamics of a pharmacologic agent, (c) characterizing the properties and therapeutic effects of a pharmacologic agent on a subject, (d) screening an experimental pharmacologic agents, and/or (e) providing implantable engineered tissues for use in regenerative medicine for treating damaged and/or diseased tissues.

Accordingly, one aspect of the present disclosure provides a method for measuring an effect of a test agent on contraction using the tissue system described herein, comprising: measuring a first value of a contraction characteristic of the 3D ex vivo skeletal muscle tissue in the bioreactor before exposure to the test agent; contacting the 3D ex vivo skeletal muscle tissue with the test agent under conditions sufficient for the test agent to modulate the contraction; measuring a second value of the contraction characteristic of the 3D ex vivo skeletal muscle tissue after exposure to the test agent; and determining whether the test agent modulates the contraction by comparing the first value with the second value.

The contraction characteristic can be a contractile force, a twitch force, a tetanic force, time to tetanus, rate of tetanic force generation, rate of tetanic force rundown, rate of relaxation from tetanic force, or a combination thereof.

In some embodiments, the test agent modulates the contraction when there is a significant difference between the first value and the second value, e.g., a difference of at least about 10%, a difference of at least about 15%, a difference of at least about 20%, a difference of at least about 25%, a difference of at least about 30%, a difference of at least about 35%, a difference of at least about 40%, a difference of at least about 45%, or a difference of at least about 50%.

In some embodiments, the measurement of a contractile force comprises measuring an amount of movement imposed by the 3D ex vivo skeletal muscle tissue on the sensing elements from a first position to a second position.

In some embodiments, the test agent modulates the contractile force when there is a significant difference between the first contractile force and the second contractile force, e.g., a difference of at least about 10%, a difference of at least about 15%, a difference of at least about 20%, a difference of at least about 25%, a difference of at least about 30%, a difference of at least about 35%, a difference of at least about 40%, a difference of at least about 45%, or a difference of at least about 50%.

In some embodiments, the contractile force can be measured by optical microscopy as disclosed by US20160282338.

Another aspect of the present disclosure relates to a method for measuring an effect of a test agent on a calcium transient using the tissue system described herein, comprising: measuring a first value of a calcium transient characteristic of the 3D ex vivo skeletal muscle tissue in the bioreactor before exposure to the test agent; contacting the 3D ex vivo skeletal muscle tissue with the test agent under conditions sufficient for the test agent to modulate the calcium transient; measuring a second value of the calcium transient characteristic of the 3D ex vivo skeletal muscle tissue after exposure to the test agent; and determining whether the test agent modulates the calcium transient by comparing the first value with the second value.

The calcium transient characteristic can be magnitude of calcium transient, time constant of calcium transient, rate of calcium transient, or a combination thereof.

In some embodiments, the test agent modulates the calcium transient when there is a significant difference between the first value and the second value, e.g., a difference of at least about 10%, a difference of at least about 15%, a difference of at least about 20%, a difference of at least about 25%, a difference of at least about 30%, a difference of at least about 35%, a difference of at least about 40%, a difference of at least about 45%, or a difference of at least about 50%.

In some embodiments, the measurement of a calcium transient characteristic comprises measuring a fluorescence signal of an intracellular calcium indicator in the 3D ex vivo skeletal muscle tissue.

In some embodiments, the intracellular calcium indicator is selected from Fura-4F AM, Fura-2, Fluo-3, Fluo-4, and Indo-1, Mag-Fura-5, and Mag-Fura-red.

Another aspect of the present disclosure relates to a method for evaluating the safety of a test agent using the tissue system described herein, comprising: (a) contacting the skeletal muscle tissue with the test agent; (b) measuring the effect on one or more physiological parameters indicative of safety; (c) comparing the physiological parameters in (b) to the same physiological parameters measured from a control bioreactor not exposed to the test agent, wherein a statistically significant change in the physiological parameters in (b) as compared to the same physiological parameters measured from the control bioreactor indicates that the test agent lacks safety.

The undesired effects of toxicity caused by administration of a test agent can be screened in several ways. The tissue system described herein can be used to determine the range of toxic dosimetry of a test agent. The effect of increasing concentrations of the test agent (i.e., dose) on skeletal muscle tissue can be monitored to detect toxicity. A toxic effect, when observed, can be equated with a measurement of test agent concentration/cells cm 2. By calculating the toxic concentration according to the distribution of cells in the skeletal muscle tissue, one of skill in the art can extrapolate to the living system, to estimate toxic doses in subjects of various weights and stages in development.

The tissue system described herein can also be used to evaluate a test agent's efficacy. Efficacy can be detected by measuring individual parameters associated with the repair, enhancement, improvement and/or regeneration of a disease model comprising a diseased skeletal muscle tissue. The diseased state can be induced or can be the result of a pre-existing condition in the tissue donor, including conditions relating to inherited genetic abnormalities. Either the induced or pre-existing condition can comprise a weakened state resulting from a previous drug exposure. Test agents can be analyzed for efficacy in disease models of the present disclosure.

Using methods of the invention, various doses of individual test agents and combinations of test agents can be screened in panels comprised of tissues having diverse genetic backgrounds to determine the pharmacogenetic efficacy profile of the test agents. For example, multiple doses of, or combinations with, test agents will be screened for efficacy, or the lack thereof, specific to one or more genetic backgrounds.

In general, test agents can be incubated with the skeletal muscle tissue in a dosage range estimated to be therapeutic and for a duration sufficient to produce an effect (e.g., metabolic effects or effects indicating to toxicity or efficacy). The incubation time can range from about one minute to 24 hours, or can be extended as necessary for several days or even weeks. The incubation conditions typically involve standard culture conditions known in the art, including culture temperatures of about 37 degrees Celsius, and culture mediums compatible with the skeletal muscle tissue.

In some embodiments, the test agent is selected from the group consisting of a small molecule, an antibody, an ion, a protein, a peptide, a lipid, DNA, RNA, a virus, bacteria, a microparticle, a nanoparticle, a therapeutic agent, and a toxin.

Examples of test agents include, but are not limited to, opioid analgesics, anti-inflammatory drugs such as antihistamines and non-steroidal anti-inflammatory drugs (NSAIDs), diuretics such as carbonic anhydrase inhibitors, loop diuretics, high-ceiling diuretics, thiazide and thiazide-like agents, and potassium-sparing diuretics, agents that impinge on the renal and cardiovascular systems such as angiotensin converting enzyme (ACE) inhibitors, cardiac drugs such as organic nitrates, calcium channel blockers, sympatholytic agents, vasodilators, β-adrenergic receptor agonists and antagonists, α-adrenergic receptor agonists and antagonists, cardiac glycosides, anti-arrhythmic drugs, agents that affect hyperlipoproteinemias such as 3-hydroxymethylglutaryl-coenzyme A (HMG-CoA) inhibitors, anti-neoplastic agents such as alkylating agents, antimetabolites, natural products, antibiotics, and other drugs, immunomodulators, anti-diabetic agents, and anti-microbial agents such as antibacterial agents, antiviral agents, antifungal agents, antiprotozoal agents, and antihelminthic agents.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.” Any ranges cited herein are inclusive.

The terms “substantially”, “approximately,” and “about” used throughout this Specification and the claims generally mean plus or minus 10% of the value stated, e.g., about 100 would include 90 to 110.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” may refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of” “only one of” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) may refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

As used herein in the specification and in the claims, the term “substantially the same” refers to a first value that is within 10% of a second value. For example, if A is substantially the same as B, and B is 100, A can have a value ranging from 90 to 110. If A is substantially the same as B, and B is 200, A can have a value ranging from 180 to 220.

As used herein, the term “POMaC” refers to poly(octamethylene maleate (anhydride) citrate) (POMaC) or the POMaC prepolymer which comprises a mixture of 1,8-octandiol, citrate acid, and maleic anhydride. Reference can be made to Tran et al., “Synthesis and characterization of a biodegradable elastomer featuring a dual crosslinking mechanism,” Soft Matter, Jan. 1, 2010; 6(11): 2449-2461, which is incorporated herein by reference in its entirety.

As used herein, the term “test agent” is any substance that is evaluated for its ability to diagnose, cure, mitigate, treat, or prevent disease in a subject, or is intended to alter the structure or function of the body of a subject. A test agent in an embodiment can be a “drug” as that term is defined under the Food Drug and Cosmetic Act, Section 321(g)(1). Test agents can include, but are not limited to, chemical compounds, biologic agents, proteins, peptides, antibodies, nucleic acids, lipids, polysaccharides, supplements, diagnostic agents and immune modulators and may also be referred to as “pharmacologic agents.”

As used herein, the term “toxicity” is defined as any unwanted effect on human cells or tissue caused by a test agent, or test agent used in combination with other pharmaceuticals, including unwanted or overly exaggerated pharmacological effects. An analogous term used in this context is “adverse reaction.”

As used herein, the term “non-human” refers to all vertebrates, e.g., mammals and non-mammals, such as nonhuman primates, sheep, dog, cat, horse, cow, chickens, amphibians, reptiles, mice, rats, and the like.

It will be understood that, although the terms “first”, “second”, etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of example embodiments. Spatially relative terms, such as “beneath,” “below,” “lower,” “above,” “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the exemplary term “below” can encompass both an orientation of above and below. The device may be otherwise oriented (rotated 90 degrees or at other orientations) and the spatially relative descriptors used herein interpreted accordingly.

The claims should not be read as limited to the described order or elements unless stated to that effect. It should be understood that various changes in form and detail may be made by one of ordinary skill in the art without departing from the spirit and scope of the appended claims. All embodiments that come within the spirit and scope of the following claims and equivalents thereto are claimed.

### EXAMPLES

### Example 1

Human Skeletal Muscle Cells (SkMCs) (catalog no. C-12530) were purchased from PromoCell at passage 2. Cells were maintained in culture in growth medium (catalog no. C23260, C-39365) and expanded by following the manufacturing instructions. Cells between passage 3 and passage 5 were used for the experiments. After detachment with trypsin/EDTA solution, SkMCs were embedded in a hydrogel with the following composition: rat tail collagen 3 mg/mL, human fibrinogen 8.2 mg/mL, Matrigel® 1:9, 50 unit/ml thrombin 1/25 of total volume. In some experiments, collagen was not used. Tissues with a cell density between 30,000 and 100,000 viable SkMCs were envisioned. In some experiments, human cardiac fibroblasts were added in a ratio 1:10. SkMCs were maintained 2 days in growth medium, followed by 1 week in differentiation medium (catalog no. C-39366 from PromoCell) and successively maintained in growth medium until the end of the study.

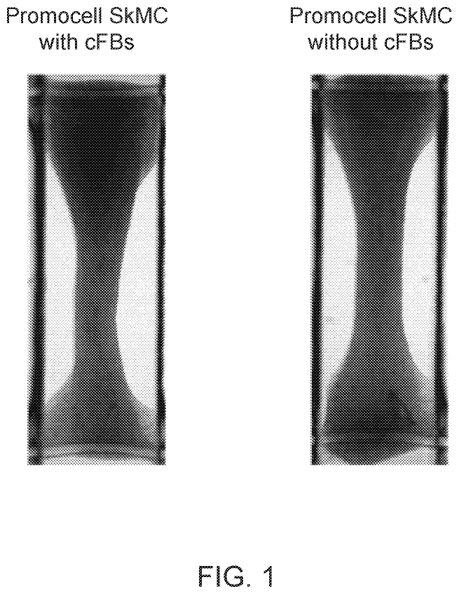

SkMC tissue compaction occurred properly, starting 24 hours after seeding and gradually proceeding overtime for 2-3 days. The presence of fibroblasts did not significantly affect tissue compaction (FIG. 1).

After 1 week of tissue seeding, external electrical stimulation was applied. Some tissues were kept unstimulated, as control, to test the hypothesis that the electrical stimulation plays a key role in skeletal tissue maturation and therefor functionality. The stimulation was started at 1 Hz with a 0.3 Hz increase/day. Media changed occurred every 2 days.

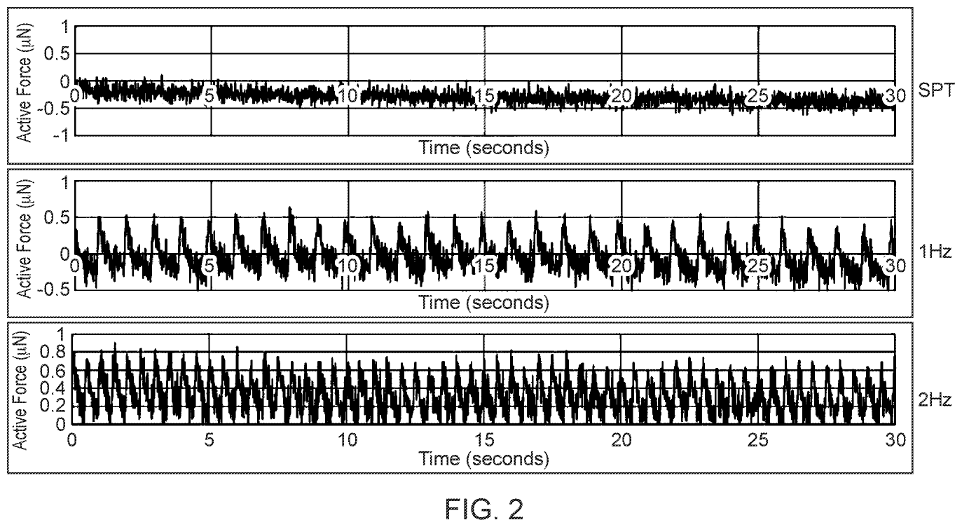

The first appearance of contraction in response to external stimulation occurred after 4 days of stimulation. No contraction was observed in unstimulated tissues (FIG. 2).

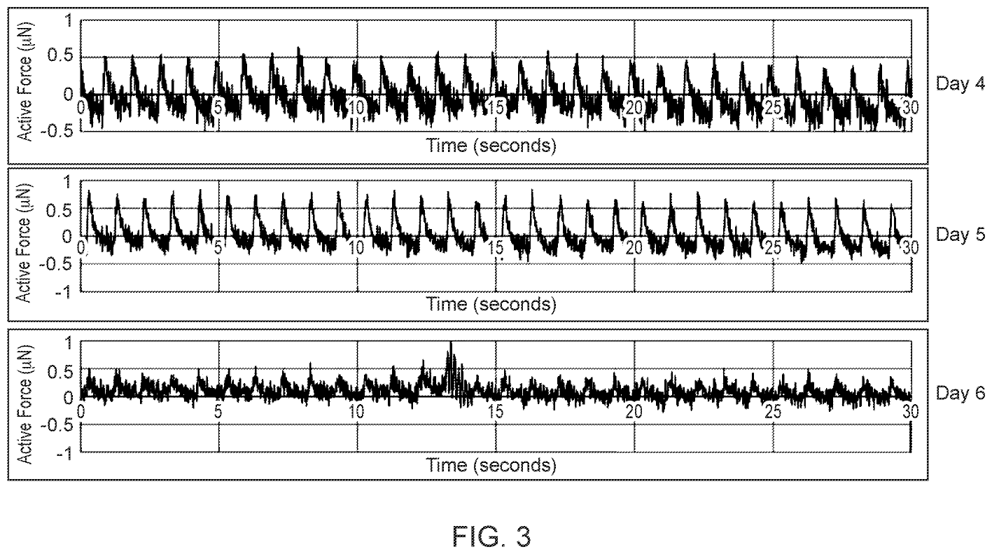

SkMC tissue contractility was analyzed overtime until day 13. No increase in active force was observed overtime (FIG. 3).

1.-20. (canceled)

21. A tissue system comprising a three-dimensional ex vivo skeletal muscle tissue and a bioreactor, wherein the three-dimensional ex vivo skeletal muscle tissue comprises a hydrogel and a plurality of cells that includes skeletal muscle cells, wherein at least a portion of the cells are encapsulated inside the hydrogel, and wherein the skeletal muscle tissue is characterized by one or more contractions in response to an electrical and/or chemical stimulation, and wherein the bioreactor comprises:
a device having a well configured for growing the three-dimensional ex vivo skeletal muscle tissue from the cells seeded therein, wherein the well has a bottom; and
at least two elastic sensing elements disposed across the well such that there is a gap between the sensing elements and the bottom of the well, wherein the sensing elements are configured to: (a) permit attachment of the three-dimensional ex vivo skeletal muscle tissue formed there between, thereby suspending the three-dimensional ex vivo skeletal muscle tissue above the bottom of the well, and (b) deform in response to a contractile force exerted on the sensing elements by the three-dimensional ex vivo skeletal muscle tissue.

22. The tissue system of claim 21, wherein the bioreactor further comprises at least two electrodes configured to apply an electrical stimulation to the three-dimensional ex vivo skeletal muscle tissue of the bioreactor.

23. The tissue system of claim 21, wherein the sensing elements comprise a synthetic polymer, a biologic polymer, or a combination thereof.

24. The tissue system of claim 23, wherein the polymer is degradable.

25. The tissue system of claim 23, wherein the polymer is nondegradable.

26. The tissue system of claim 21, wherein the at least two elastic sensing elements comprise a polymer selected from the group consisting of polylactic acid, poly(lactic-co-glycolic) acid, poly(caprolactone), polyglycolide, polylactide, polyhydroxobutyrate, polyhydroxyalcanoic acid, chitosan, hyaluronic acid, poly(2-hydroxyethyl-methacrylate), poly(ethylene glycol), poly(L-lactide) (PLA), poly(dimethysiloxane) (PDMS), poly(methylmethacrylate) (PMMA), poly(glycerol sebacate), poly(octamethylene maleate (anhydride) citrate) (POMaC), POMaC without citric acid, poly(£-caprolactone), polyurethane, silk, and a combination thereof.

27. The tissue system of claim 21, wherein the polymer comprises POMaC.

28. The tissue system of claim 21, wherein the sensing elements have an elasticity from about 10 kPa to 0.8 MPa.

29. The tissue system of claim 21, wherein the at least two elastic sensing elements are in the form of polymer wires.

30. The tissue system of claim 21, wherein the bioreactor comprises 2 to 25 sensing elements per well.

31. The tissue system of claim 21, wherein the bioreactor comprises a multi-well plate.

32. The tissue system of claim 31, wherein the multi-well plate comprises 6 wells, 8 wells, 12 wells, 24 wells, 96 wells, 384 wells, or 1536 wells.

33. A method for measuring an effect of a test agent on contraction using the tissue system of claim 21, comprising:
measuring a first value of a contraction characteristic of the three-dimensional ex vivo skeletal muscle tissue in the bioreactor before exposure to the test agent;
contacting the three-dimensional ex vivo skeletal muscle tissue with the test agent under conditions sufficient for the test agent to modulate the contraction;
measuring a second value of the contraction characteristic of the three-dimensional ex vivo skeletal muscle tissue after exposure to the test agent; and
determining whether the test agent modulates the contraction by comparing the first value with the second value.

34. The method of claim 33, wherein the test agent modulates the contraction when there is a significant difference between the first value and the second value.

35. The method of claim 33, wherein the test agent is selected from the group consisting of a small molecule, an antibody, an ion, a protein, a peptide, a lipid, DNA, RNA, a virus, bacteria, a microparticle, a nanoparticle, a therapeutic agent, and a toxin.

36. A method for measuring an effect of a test agent on a calcium transient using the tissue system of claim 21, comprising:
measuring a first value of a calcium transient characteristic of the three-dimensional ex vivo skeletal muscle tissue in the bioreactor before exposure to the test agent;
contacting the three-dimensional ex vivo skeletal muscle tissue with the test agent under conditions sufficient for the test agent to modulate the calcium transient;
measuring a second value of the calcium transient characteristic of the three-dimensional ex vivo skeletal muscle tissue after exposure to the test agent; and
determining whether the test agent modulates the calcium transient by comparing the first value with the second value.

37. The method of claim 36, wherein measuring the first value or second value comprises measuring a fluorescence signal of an intracellular calcium indicator in the three-dimensional ex vivo skeletal muscle tissue.

38. The method of claim 37, wherein the intracellular calcium indicator is selected from Fura-4F AM, Fura-2, Fluo-3, Fluo-4, and Indo-1, Mag-Fura-5, and Mag-Fura-red.

39. The method of claim 36, wherein the test agent modulates the calcium transient when there is a significant difference between the first calcium transient and the second calcium transient.

40. The method of claim 36, wherein the test agent is selected from the group consisting of a small molecule, an antibody, an ion, a protein, a peptide, a lipid, DNA, RNA, a virus, bacteria, a microparticle, a nanoparticle, a therapeutic agent, and a toxin.
